(19) United States
(12) Patent Application Publication
(10) Pub. No.: US 2008/0085882 A1
(43) Pub. Date: Apr. 10, 2008

(54) Compositions and Methods for Potentiation of Cancer Agents

(75) Inventors: Wolfram Siede, Benbrook, TX (US); Rouel S. Roque, Fort Worth, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 11/840,849

(22) Filed: Aug. 17, 2007

Related U.S. Application Data
(60) Provisional application No. 60/838,538, filed on Aug. 18, 2006.

(51) Int. Cl. A61K 31/555; A61K 31/5375; A61P 35/00

(52) U.S. Cl. 514/185; 514/229.5

(57) ABSTRACT

The present invention includes compositions and method to improve the therapeutic index of anti-cancer agents using a novel anti-cancer agent and a modulator or potentiator thereof.

### CROSS-REFERENCE TO RELATED APPLICATIONS

This application claims priority to U.S. Provisional Patent Application Ser. No. 60,868,538, filed Aug. 18, 2006, the entire contents of which are incorporated herein by reference. This application is related to U.S. Provisional Patent Application Ser. No. 60,868,567 and filed Aug. 18, 2006, the entire contents of which is also incorporated herein by reference.

### STATEMENT OF FEDERALLY FUNDED RESEARCH

This invention was made with U.S. Government support under Contract No. RO1 CA087381 awarded by the National Cancer Institute at the National Institutes of Health. The government has certain rights in this invention.

### TECHNICAL FIELD OF THE INVENTION

The present invention relates in general to the field of cancer therapy modulators, and more particularly, to the use of agents that include copper to improve the therapeutic index of anti-cancer agents.

### BACKGROUND OF THE INVENTION

Without limiting the scope of the invention, its background is described in connection with the treatment of cancer.

In various settings, human cells are exposed to DNA-damaging agents. Such exposure frequently amounts to a significant health risk. Examples include exposure to chemicals or radiation from various sources, e.g., due to environmental contamination, sun light, space travel, medical diagnostic procedures or catastrophic events (chemical warfare, nuclear accidents, terrorism).

On the other hand, in cancer therapy, such exposure can be beneficial provided there is a precise targeting of tumor cells and/or a sufficient difference in sensitivity between tumor cells and normal cells (“therapeutic window” or “index”). Depending on the scenario, there is a need for protection of non-tumor cells or for increasing the sensitivity of tumor cells in order to enhance the therapeutic index.

### SUMMARY OF THE INVENTION

The present invention includes compositions and methods for the treatment of cancers by combining a primary active agent and a modulator of that agent. More particularly, the present invention includes a pharmaceutical composition that includes an chemotherapeutic agent and an antitumor modulator that is a bis(3-amino-1-alkanolato-)-di-μ-[halo/hydroxyl/thiocyanato/nitro]-di-Copper complex and derivatives thereof, wherein the chemotherapeutic agent is provided at an optimal or sub-optimal dose. The modulator may be a potentiator provided in the form of a pharmaceutically acceptable salt thereof and/or a prodrug thereof. The composition may be micronized and made suitable for administration to the warm blooded animal by injection. For example, the composition may include an otherwise optimal or sub-optimal doses of the chemotherapeutic agent, e.g., in an amount of from 10 mg/kg body weight to 10,000 mg/kg body weight for the active agent in conjunction with the potentiator to enhance the anti-tumor effect of the active agent in the composition, while maintaining a reduced side-effect profile. The composition may be provided by any of the many known methods and administrated, e.g., orally, enterically, intravenously, peritoneally, parenterally, subcutaneously, or by injection, to name a few. The composition will most often be administered in a pharmaceutically acceptable carrier. Alternatively, the composition of the present invention may also include a safe and effective amount of a second chemotherapeutic agent.

Examples of cancer cells that may be treated with the composition and/or potentiator of the present invention include, e.g., astrocytoma, glioblastoma carcinoma, leukemia, melanoma, colon cancer, breast cancer, lung cancer, brain cancer, pancreatic cancer, ovarian cancer, head and neck cancer, liver cancer, and prostate cancer. Other non-limiting examples of cancers include, e.g., astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

The present invention also includes methods for treating cancer susceptible to treatment in a warm-blooded animal comprising administering to the warm-blooded animal a therapeutically effective amount of a DNA damaging agent and a potentiator comprising at least one of:

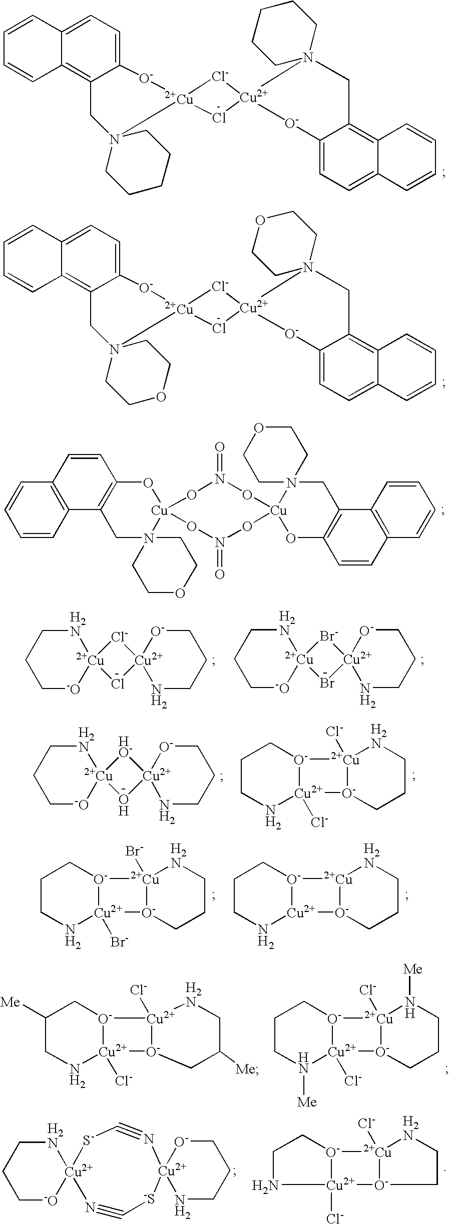

The potentiator is generally provided in the form of one or more pharmaceutically acceptable salt thereof.

The present invention also includes a method for treating a warm-blooded animal susceptible to DNA damage comprising administering to the warm-blooded animal a therapeutically effective amount of a bis(3-amino-1-alkanolato-)-di-μ-[halo/hydroxyl/thiocyanato/nitro]-di-Copper complex, e.g., NSC109268. Examples of the potentiator include: Copper, di-μ-chlorobis[1-[(1-piperidinyl-κN)methyl]-2-naphthalenolato-κO]di-(9CI), Copper, di-μ-chlorobis[1-[(4-morpholinyl-κN4)methyl]-2-naphthalenolato-κO]di-(9CI), Copper, di-μ-nitrate bis[1-[(4-morpholinyl-κN4)methyl]-2-naphthalenolato-κO]di-(9CI), Copper, bis(3-amino-1-propanolato-N,O)di-μ-chlorodi-(9CI), Copper, bis(3-amino-1-propanolato-N,O)di-μ-bromodi-(9CI), Copper, bis(3-amino-1-propanolato-N,O)di-μ-hydroxydi-(9CI), Copper, bis[μ-(3-amino-1-propanolato-N,O:O)]dichlorodi-, stereoisomer (9CI), Copper, bis[μ-(3-amino-1-propanolato-N,O:O)]dibromodi-, stereoisomer (9CI), Copper, bis[μ-(3-amino-2-methyl-1-propanolato-N,O:O)]dichlorodi-, stereoisomer (9CI), Copper, bis[μ-(3-amino-2-methyl-1-propanolato-N,O:O)]dichlorodi-, stereoisomer (9CI), Copper, dichlorobis[μ-[3-(methylamino)-1-propanolato-N,O:O]]di-(9CI), Copper, bis(3-amino-1-propanolato-N,O)bis[μ-(thiocyanato-N:S)]di-(9CI), and Copper, bis[μ-(2-aminoethanolato-N,O:O)]dichlorodi-, stereoisomer (9CI).

Examples of DNA damage include those caused by chemical, ultraviolet light, heat, radioactivity, electromagnetic radiation, electricity and combinations thereof. In one specific, example the warm-blooded animals are mammals such as humans, dogs, cats, horses, cows, pigs, monkeys, goats and the like.

The present invention also includes compositions and methods for improving the therapeutic index of a chemotherapeutic agent by providing a therapeutically effective amount of NSC109268 to potentiate the anti-tumor activity of the chemotherapeutic agent. Examples of tumors include, e.g., astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that demonstrates the effect of cisplatin and NSC109268 on carcinoma cell line PC3 and shows that NSC109268 increases cellular sensitivity to cisplatin in a range of concentrations where NSC109268 is (virtually) non-toxic.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “modulator” refers to the biologic activity of a bis(3-amino-1-alkanolato-)-di-μ-[halo/hydroxyl/thiocyanato/nitro]-di-Copper complex, its derivatives, precursors, metabolites or mixtures thereof, wherein the bis(3-amino-1-alkanolato-)-di-μ-[halo/hydroxyl/thiocyanato/nitro]-di-Copper complex interacts with a target and increases or decreases the activity of the chemotherapeutic agent. The interactions include, but are not limited to, agonist, antagonist, and the like. In fact, in some cases the therapeutic index may be decreased if the modulator of the present invention also decreases side-effects without affecting potency of the anti-cancer agent.

As used herein, the term “alkanolato” refers to an alkyl, cycloalkyl, or aryl organic compound containing the —C(—OH)— group, where the H of the —OH group has been replaced by a metal.

As used herein, the term “potentiator” refers to a compound that accentuates or potentiates a cytotoxic activity upon a cell, e.g., a cell that is contacted with the bis(3-amino-1-alkanolato-)-di-μ-[halo/hydroxyl/thiocyanato/nitro]-di-Copper complex, a derivative, precursor, a metabolite or a mixture thereof in conjunction with an optimal or sub-optimal dose of DNA damaging agent. Non-limiting examples of the potentiator of the present invention include: Copper, di-μ-chlorobis[1-[(1-piperidinyl-κN)methyl]-2-naphthalenolato-κO]di-(9CI), Copper, di-μ-chlorobis[1-[(4-morpholinyl-κN4)methyl]-2-naphthalenolato-κO]di-(9CI), Copper, di-μ-nitrate bis[1-[(4-morpholinyl-κN4)methyl]-2-naphthalenolato-κO]di-(9CI), Copper, bis(3-amino-1-propanolato-N,O)di-μ-chlorodi-(9CI), Copper, bis(3-amino-1-propanolato-N,O)di-μ-bromodi-(9CI), Copper, bis(3-amino-1-propanolato-N,O)di-μ-hydroxydi-(9CI), Copper, bis[μ-(3-amino-1-propanolato-N,O:O)]dichlorodi-, stereoisomer (9CI), Copper, bis[μ-(3-amino-1-propanolato-N,O:O)]dibromodi-, stereoisomer (9CI), Copper, bis[μ-(3-amino-2-methyl-1-propanolato-N,O:O)]dichlorodi-, stereoisomer (9CI), Copper, bis[μ-(3-amino-2-methyl-1-propanolato-N,O:O)]dichlorodi-, stereoisomer (9CI), Copper, dichlorobis[μ-[3-(methylamino)-1-propanolato-N,O:O]]di-(9CI), Copper, bis(3-amino-1-propanolato-N,O)bis[μ-(thiocyanato-N:S)]di-(9CI), and Copper, bis[μ-(2-aminoethanolato-N,O:O)]dichlorodi-, stereoisomer (9CI), derivatives, precursors, metabolites and combinations thereof.

The potentiator may be added, admixed, co-administered, administered in series or in parallel with an optimal or sub-optimal dose of one or more DNA damaging agents, another potentiator and/or another chemotherapeutic agent. The potentiator may be added before, during or after a dose of an optimal or sub-optimal doses of DNA damaging agents, another potentiator and/or another chemotherapeutic agent and may even be conjugated directly with the one or more agents, either covalently or ionically. In one example, the potentiator is mixed with an optimal or sub-optimal doses of DNA damaging agents, another potentiator and/or another chemotherapeutic agent in a biodegradable resin or matrix that releases the active agents at the same or different rates, at the same or disparate times and combinations thereof.

Examples of the potentiator of the present invention include:
Registry Number: 691005-38-6

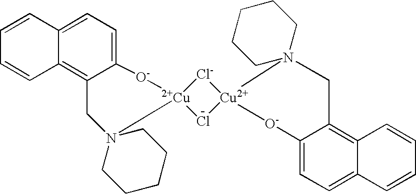

Formula: C32 H36 C12 Cu2 N2 O2

CA Index Name: Copper, di-μ-chlorobis[1-[(1-piperidinyl-κN)methyl]-2-naphthalenolato-κO]di-(9CI)

Other Names: NSC 109268

Registry Number: 881886-17-5

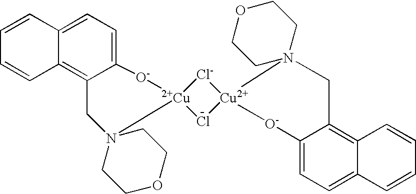

Formula: C30 H32 C12 Cu2 N2 O4

Other Names: NSC 109272, in additional the following compound (not depicted) may also be used as a potentiator: NSC 109271, which differs from 109272 in that 109271 (structure not shown) that is similar to this one except that two nitrate ions (NO₃) that bridge the Cu²⁺ ions.

CA Index Name: Copper, di-μ-chlorobis[1-[(4-morpholinyl-κN4)methyl]-2-naphthalenolato-κO]di-(9CI)

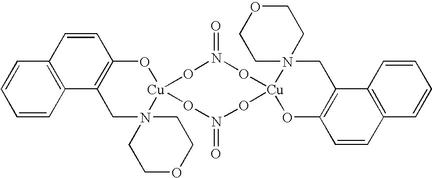

Also known as NSC 109271, which differs from 109272 in that 109271 (structure not shown) that is similar to this one except that two nitrate ions (NO₃) that bridge the Cu²⁺ ions.

CA Index Name: Copper, di-μ-nitrate bis[1-[(4-morpholinyl-κN4)methyl]-2-naphthalenolato-κO]di-(9CI)

Registry Number: 52564-68-8

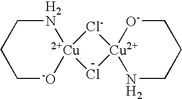

Formula: C6 H16 C12 Cu2 N2 O2

CA Index Name: Copper, bis(3-amino-1-propanolato-N,O)di-μ-chlorodi-(9CI)

Other Names: 1-Propanol, 3-amino-, copper complex

Registry Number: 52564-69-9

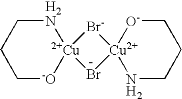

Formula: C6 H16 Br2 Cu2 N2 O2

CA Index Name: Copper, bis(3 -amino-1-propanolato-N,O)di-μ-bromodi-(9CI)

Other Names: 1-Propanol, 3-amino-, copper complex

Registry Number: 52563-76-5

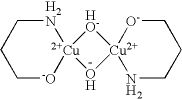

Formula: C6 H18 Cu2 N2 O4

CA Index Name: Copper, bis(3-amino-1-propanolato-N,O)di-μ-hydroxydi-(9CI)

Other Names: 1-Propanol, 3-amino-, copper complex

Registry Number: 87464-83-3

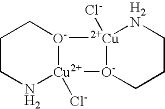

Formula: C6 H16 C12 Cu2 N2 O2

CA Index Name: Copper, bis[μ-(3-amino-1-propanolato-N,O:O)]dichlorodi-, stereoisomer (9CI)

Other Names: 1-Propanol, 3-amino-, copper complex

Registry Number: 87464-84-4

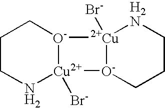

Formula: C6 H16 Br2 Cu2 N2 O2

CA Index Name: Copper, bis[μ-(3-amino-1-propanolato-N,O:O)]dibromodi-, stereoisomer (9CI)

Other Names: 1-Propanol, 3-amino-, copper complex

Registry Number: 851428-54-1

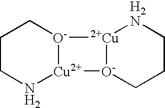

Formula: C6 H16 Cu2 N2 O2

CA Index Name: Copper(2+), bis[μ-[3-(amino-κN)-1-propanolato-κO:κO]]di-(9CI)

Registry Number: 87464-85-5

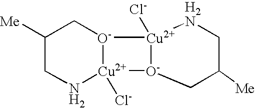

Formula: C8 H20 C12 Cu2 N2 O2

CA Index Name: Copper, bis[μ-(3-amino-2-methyl-1-propanolato-N,O:O)]dichlorodi-, stereoisomer (9CI)

Other Names: 1-Propanol, 3-amino-2-methyl-, copper complex

Registry Number: 89172-58-7

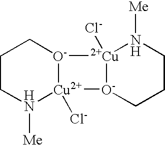

Formula: C8 H20 C12 Cu2 N2 O2

CA Index Name: Copper, dichlorobis[μ-[3-(methylamino)-1-propanolato-N,O:O]]di-(9CI)

Other Names: 1-Propanol, 3-(methylamino)-, copper complex

Registry Number: 52563-78-7

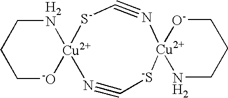

Formula: C8 H16 Cu2 N4 O2 S2

CA Index Name: Copper, bis(3-amino-1-propanolato-N,O)bis[μ-(thiocyanato-N:S)]di-(9CI)

Other Names: 1-Propanol, 3-amino-, copper complex

Registry Number: 166531-85-7

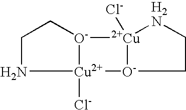

Formula: C4 H12 C12 Cu2 N2 O2

CA Index Name: Copper, bis[μ-(2-aminoethanolato-N,O:O)]dichlorodi-, stereoisomer (9CI)

As used herein, a “pharmaceutical salts” is salt for making an acid or base salts of a compounds. Examples of pharmaceutically acceptable salts include, but are not limited to, mineral or organic acid salts of basic residues such as amines; alkali or organic salts of acidic residues such as phenols. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The preferred phenolate salts are the alkaline earth metal salts, sodium, potassium or lithium.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “pharmaceutical carrier” is a pharmaceutically acceptable solvent, suspending agent or vehicle, for delivering an optimal or sub-optimal doses of DNA damaging agents and the potentiator of the present invention and/or another chemotherapeutic agent to the animal or human. The carrier may be liquid or solid and is selected with the planned manner of administration in mind. Liposomes are also a pharmaceutical carrier.

As used herein, a “subject in need thereof” is a warm blooded animal having cancer or having been exposed to an agent, ray or emission that causes DNA damage.

As used herein, “cancer” refers to all types of cancers, or neoplasms or benign or malignant tumors. In one embodiment, those cancers that attack normal healthy blood cells or bone marrow are contemplated by the present invention. Examples of cancers for treatment using methods provided herein include glioblastoma, melanoma, carcinoma, sarcoma, lymphoma, or leukemia. By “carcinoma” is meant a benign or malignant epithelial tumor and includes, but is not limited to, breast carcinoma, prostate carcinoma, non-small cell lung carcinoma, colon carcinoma, CNS carcinoma, melanoma carcinoma, ovarian carcinoma, or renal carcinoma. Examples of cancers in certain tissues are cancer of the brain, breast, cervix, colon, head & neck, kidney, lung, non-small cell lung, melanoma, mesothelioma, ovary, sarcoma, stomach, uterus and medulloblastoma. The cancer may form a tumor, e.g., astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of: (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with optimal or sub-optimal doses of DNA damaging agents and the potentiator of the present invention include, e.g., a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectatic sarcoma.

The term “melanoma” is taken to mean a tumor arising from the melanocytic system of the skin and other organs. Melanomas which can be treated with an optimal or sub-optimal doses of DNA damaging agents and the potentiator of the present invention and/or another chemotherapeutic agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with an optimal or sub-optimal doses of DNA damaging agents and the potentiator of the present invention and/or an additional chemotherapeutic agent include, e.g., acinar carcinoma, acinous carcinoma, adenocystic carcinoma, adenoid cystic carcinoma, carcinoma adenomatous, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

As used herein, the term “DNA damaging agents,” refers to those chemotherapeutic agents and/or radiation (e.g., U.V., gamma radiation, etc.) that directly or indirectly cause DNA-damage and/or activation of one or more DNA repair mechanisms. Chemotherapeutic agents used in combination with the potentiator of the present invention or salts thereof may be selected from any of these groups but are not limited thereto. For a detailed discussion of the chemotherapeutic agents that may be used in conjunction with the present invention and their dosage and method of administration, see, e.g., Dorr, et al., The Cancer Chemotherapy Handbook (6th Edition) by Fischer, Durivage, Knobf, and Beaulieu, Mosby-Year Book, Inc. (2003), relevant dosage forms, treatment regimes and handling requirements incorporated herein by reference.

DNA-interactive agents include alkylating agents, e.g., cisplatin, cyclophosphamide, and altretamine; DNA strand-breakage agents, such as bleomycin; intercalating topoisomerase II inhibitors, e.g., dactinomycin and doxorubicin; nonintercalating topoisomerase II inhibitors, such as etoposide and teniposide; and the DNA minor groove binder plicamycin, for example. The alkylating agents form covalent chemical adducts with cellular DNA, RNA, or protein molecules, or with smaller amino acids, glutathione, or similar chemicals. Generally, alkylating agents react with a nucleophilic atom in a cellular constituent, such as an amino, carboxyl, phosphate, or sulfhydryl group in nucleic acids, proteins, amino acids, or in glutathione.

Typical alkylating agents include, but are not limited to, nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. DNA strand breaking agents include bleomycin, for example. DNA topoisomerase II inhibitors include the following intercalators, such as amsacrine, dactinomycin, daunorubicin, doxorubicin (adriamycin), idarubicin, and mitoxantrone; nonintercalators, such as etoposide and teniposide, for example. A DNA minor groove binder is plicamycin, for example. Antimetabolites interfere with the production of nucleic acids by one of two major mechanisms. Certain drugs inhibit production of deoxyribonucleoside triphosphates that are the immediate precursors for DNA synthesis, thus inhibiting DNA replication. Certain of the compounds are analogues of purines or pyrimidines and are incorporated in anabolic nucleotide pathways. These analogues are then substituted into DNA or RNA instead of their normal counterparts.

Other examples of agents for use with the present invention include Melphalan, which is used to treat breast cancer, ovarian cancer, multiple myeloma, melanoma, brain cancer, and leukemia, and may cross-links strands of DNA and riboneucleic acid, and inhibits protein synthesis. Melphalan works in rapidly dividing cancer cells as well as those at rest and is most commonly used drugs in limb perfusion. Side effects of Melphalan include dermatitis, nausea, vomiting, and bone marrow depression. Generally, it is expected that other phenylalanine derivative of nitrogen mustard, such as Melphalan will also work in conjunction with the potentiator of the present invention. Epidemiological studies of patients with ovarian carcinoma, multiple myeloma, or breast cancer have consistently shown very large excesses of acute nonlymphocytic leukemia in the decade following therapy with melphalan. The relative risk was consistently estimated to be in excess of 100, to increase with increasing dose. Patients treated therapeutically with melphalan had increased frequencies of chromosomal aberration and sister chromatid exchanges in their peripheral lymphocytes. Melphalan provides a cost-effective regiments for ovarian cancer and may be used in high dose chemotherapy trials as well as treatment of Cisplatin resistant cells.

Yet another agent for use in conjunction with the potentiator of the present invention is Trichlormethine, which is a nitrogen mustard used in the treatment of leukemia and lymphoma since 1946. Another agent is Gallic Acid, which is a Histamine inhibitor, Proinflammatory cytokine. Gallic acid may also have antiviral and antifungal properties and act as an antioxidant.

A two-hybrid assay-based yeast system was used to screen small-molecule compound libraries for agents that modify cell cycle checkpoint activation. Checkpoint activation is measured as the DNA-damaged-induced increased interaction between two selected checkpoint proteins. From a 1,900 small-molecule library provided by the Developmental Therapeutics Branch of the National Cancer Institute, compound NSC109268 was found to interfere with checkpoint activation in response to the topoisomerase I inhibitor camptothecin. Closer inspection of NSC109268's activity suggests that its mode of action does most likely not reflect a specific effect on checkpoint signaling; rather, it appears to protect yeast cells from the lethal effect of certain DNA damaging agents.

As another example, the drug streptonigrin was used as a double-strand break inducing drug. After treatment of logarithmically growing yeast cultures for 1 h at 30° C. in YPD medium (1% yeast extract, 2% peptone, 2% dextrose), lethality was measured by inability of colony formation on YPD agar plates. A protective effect of NSC109268 was evident and most visible in a rad5l mutant, defective in double-strand break repair by homologous recombination. The compound itself exhibited moderate toxicity. The used concentration of 50-100 μg/ml results in 80% survival after 1 h incubation. Similar protective effects were found with an alkylating agent (MNNG) and, less significantly, also with ionizing radiation (¹³⁷Cs γ-radiation). The compound does not seem to affect sensitivity to another alkylating agent (MMS), to ultraviolet radiation (UV-C) or to phleomycin (see Table 1).

Curiously, the opposite effect was found for other agents such hydrogen peroxide (H₂O₂), an oxidative agent causing base damage and strand breaks. Here, sensitivity is enhanced by simultaneous exposure to NSC109268. A similar observation was made with the anticancer drug cisplatin (cis-Diammineplatinum (II) dichloride, Sigma-Aldrich Corp., Cat. No. P4394).

Not wanting to be bound by theory, NSC109268 may protect from or facilitate the initial introduction of damage, may inhibit or facilitate DNA repair or may increase or reduce lethal consequences of DNA damage. An influence on cell cycle progression that may be critical for these effects was also observed. Furthermore, the chemistry of NSC109268 suggests possible intercalation into DNA. The Cu²⁺ ions that are bound may influence radical emergence close to DNA and modify the efficiency of agents that rely on radical production.

In summary, the incubation of logarithmic phase cells of wild-type yeast and a double-strand break repair deficient derivative (rad51) (Amberg, D. C., D. J. Burke, and J. N. Strathern. 2005. Methods in Yeast Genetics: A Cold Spring Harbor Laboratory Course Manual, 2005 Edition. Cold Spring Harbor Laboratory Press, Cold Spring Harbor, N.Y.) with compound NSC109268 had the following effects on sensitivity towards DNA damaging agents, measured as survival of macrocolony forming cells (+increased resistance, −decreased resistance, ND not determined): Table 1 summarizes the effects of NSC109268 on senstivity towards DNA Damaging Agents in repair-proficient wild type and rad 51⁻ cells.

TABLE 1

| DNA damaging agent | strain WT | strain rad51 |
|---|---|---|
| Streptonigrin | + | +++ |
| MNNG | + | +++ |
| MMS | ND | +/− |
| Phleomycin | +/− | + |
| Camptothecin | (+) | ++ |
| Ionizing radiation (¹³⁷Cs) | (+) | + |
| Ultraviolet light | +/− | +/− |
| H₂O₂ | ND | −− |
| Cisplatin | − | − |

This enhancement of cisplatin sensitivity by NSC109268 was quantified. We also tested the chemically related compound NSC 109272. Cells of a logarithmic-phase culture of WT strain BY4741 were incubated for 2 hours at 30° C. in water containing the combinations of NSC109268 or NSC109272 and cisplatin shown in Table 2. The fraction of colony forming cells was determined on solid YPD medium.

TABLE 2
Effect of cisplatin/NSC109268 or NSC109272 combination
treatments on cell survival

| | | NSC109268 | NSC109268 | NSC109272 |
|---|---|---|---|---|
| | 0 | 7.4 nM | 37 nM | 1.5 nM |
| 0 μM | 100% | 79.5% | 10% | 70% |
| Cisplatin | | | | |
| 33 μM | 71% | 29% | ND | ND |
| Cisplatin | | | | |
| 82.5 μM | 44.5% | 25.5% | ND | ND |
| Cisplatin | | | | |
| 165 μM | 21% | 0.09% | <0.4% | 4% |
| Cisplatin | | | | |

Based on the cisplatin result in yeast, the inventors explored if a similar effect can be detected in human cells. RWPE-1 normal prostate cell line (provided by J. Rhim, Uniformed Services University of the Health Sciences, Bethesda, Md.), PC3 prostate adenocarcinoma cells (American Type Culture Collection, Manassas, Va.), cisplatin sensitive ovarian cancer cells (2008) and cisplatin resistant ovarian cancer cells (2008/IC3) (provided by S. B. Howell, University of California, San Diego) were treated with cisplatin and varying amounts of NSC109268 to demonstrate the potentiation of cisplatin by NSC109268. Survival was determined in the presence or absence of compound NSC109268 using Cell Titer 96 Aqueous Cell Proliferation Assay kit from Promega, Madison, Wis. All cultures, unless specified otherwise, were maintained in growth medium composed of RPMI with 2 mM L-glutamine, 100 units/ml of Penicillin, and 100 μg/ml Streptomycin, supplemented with 10% fetal bovine serum under a humidified atmosphere of 95% air, 5% CO₂ at 37° C.

Briefly, cells were plated in 96-well tissue culture plates at 5-8×10³ cells/well and allowed to attach overnight in growth medium. The following day, cells were rinsed twice in DMEM and further incubated in serum-free medium for 24 h. Cells were then treated with cisplatin [0-100 μM] in the presence of NSC109268 [0, 1, 2, 3 μM] for 48 h. Dose-response studies were initially done to determine appropriate dosages for the drugs and their vehicles. Negative controls involved the use of DMF, vehicle for cisplatin (˜0 μM) or DMSO, vehicle for NSC109268 (˜0 μM).

To determine the number of surviving cells, cells were incubated in 333 μg/ml MTS [3-(4,5-dimethylthiazol-2-yl)-5-(3-carboxymethoxyphenyl)-2-(4-sulfophenyl)-2H-tetrazolium], a tetrazolium salt, and 25 μM phenazine methosulfate (PMS), an electron coupling reagent. MTS is taken up by live cells where it is reduced by the dehydrogenase enzymes and released back into the culture medium as a yellow formazan product. The amount of formazan product measured in terms of absorbances is directly proportional to the number of live cells in the cultures. Thus, absorbance readings at 490 nm were taken after 1 h and converted to cell numbers based on standard curves. Standard curves were done using cells maintained in RPMI in 96-well plates at the same time. Experiments were done in triplicate wells at least 4 times. Statistical analyses were done using ANOVA.

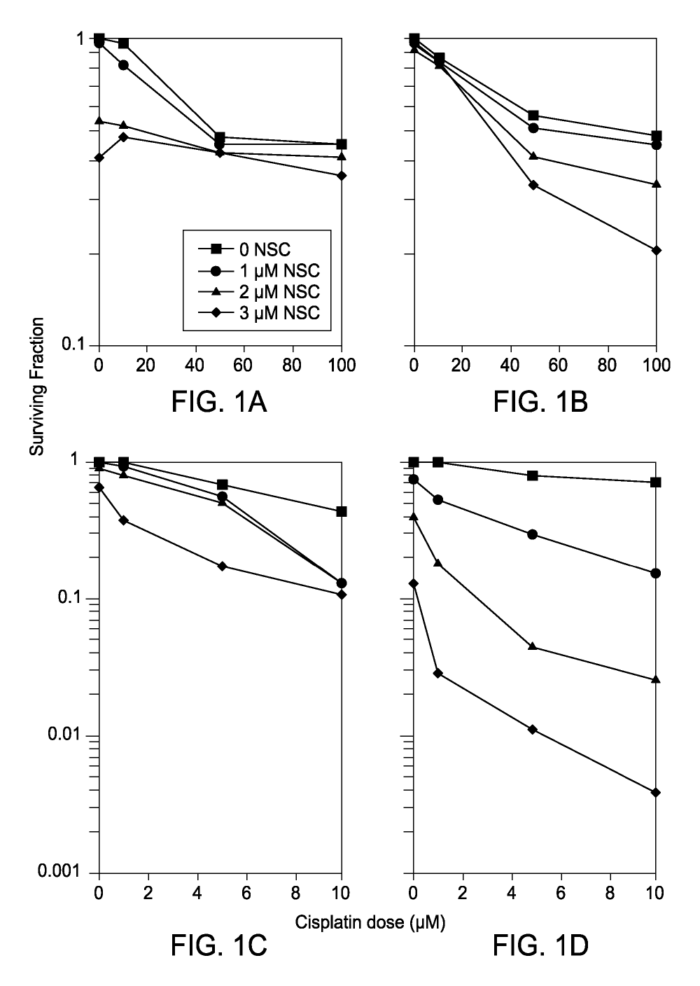

FIG. 1 is a graph that shows the effect of NSC109268 on RWPE-1 normal prostate cell line as well as prostate and ovarian tumor cells. Several series of studies were conducted using DMF (solvent control=0) and various concentrations of cisplatin in combination with (DMSO (solvent control=0), 1 μM NSC1092682 μM NSC109268, and 3 μM NSC109268. Survival was plotted in a logarithmic scale against cisplatin dose, with different symbols indicating the amount of added NSC109268 (see FIG. 1A insert), for the RWPE-1 normal prostate cell line (FIG. 1A), PC3 prostate cancer cells (FIG. 1B), cisplatin-sensitive ovarian cancer cells (2008) (FIG. 1C) and cisplatin-resistant ovarian cancer cells (2008/IC3) (FIG. 1D).

It was found that in all cases except normal prostate cells a combination of cisplatin and NSC doses can be defined where the effects on survival are synergistically enhanced as expected for a potentiator and cannot be explained by additivity of individual effects. For example, 5 μM of cisplatin without any added NSC109268 results in 85% survival, 2 μM NSC109268 without any cisplatin results in 38% survival in cisplatin resistant ovarian cancer cells (FIG. 1D). If the effects were independent, survival of the 5 μM cisplatin/2 μM NSC10928 combination treatment should be 85% of 38% and thus 32%. The measured survival, however, is 4.5% and thus much lower. NSC10928 thus potentiates the cisplatin effect.

It was also noted that the enhancement effect in cisplatin-resistant cells (2008/IC3) is much more pronounced as compared to cisplatin-sensitive cells (compare FIG. 1C and D). In fact, the combination treatment renders cisplatin-resistant cells to be more sensitive than cis-platin sensitive cells. Additionally, NSC109268 without added cisplatin is much more toxic to cisplatin-resistant cells. The emergence of cisplatin-resistant tumor cells within an initially cisplatin-sensitive tumor is an important problem of ovarian cancer therapy (Siddik Z H, Oncogene 22 [2003] 7265-79). Based on the results shown in FIG. 1, it was found that a combination treatment (or even a treatment with NSC alone) will effectively and preferentially eliminate cisplatin resistant cells from a mixed tumor cell population.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Bartel, P. L., and S. Fields. 1995. Analyzing protein-protein interactions using 2-hybrid system. Meth. Enzymol. 254:241-263.

Busby, E. C., D. F. Leistritz, R. T. Abraham, L. M. Karnitz, and J. N. Sarkaria. 2000. The radiosensitizing agent 7-hydroxystaurosporine (UCN-01) inhibits the DNA damage checkpoint kinase hChk1. Cancer Res. 60:2108-2112.

Chu, G. 1994. Cellular responses to cisplatin. J. Biol. Chem. 269:787-790.

Daniel, K. G., P. Gupta, R. H. Harbach, W. C. Guida, and Q. P. Dou. 2004. Organic copper complexes as a new class of proteasome inhibitors and apoptosis inducers in human cancer cells. Biochem. Pharmacol. 67:1139-1151.

Friedberg, E. C., G. C. Walker, W. Siede, R. D. Wood, R. A. Schultz, and T. Ellenberger. 2005. DNA Repair and Mutagenesis, 2nd Edition. American Society of Microbiology Press, Washington, D.C.

Holbeck, S. L. 2004. Update on NCI in vitro drug screen utilities. Eur. J. Cancer 40:785-793.

Rogers, J. P., A. E. Beuscher IV, M. Flajolet, T. McAvoy, A. C. Nairn, A. J. Olson, and P. Greengard. 2006. Discovery of protein phosphatase 2C by virtual screening. J. Med. Chem. 49:1658-1667.

Sedletska, Y., M. Giraud-Panis, and J. Malinge. 2005. Cisplatin is a DNA-damaging antitumour compound triggering multifactorial biochemical responses in cancer cells: importance of apoptotic pathways. Curr. Med. Chem.-Anti-Cancer Agents 5:251-265.

Sharma, V., Peddibhotia, S., and Tepe, J. J., Sensitization of cancer cells to DNA damaging agents by imidazolines J. Am. Chem. Soc. 128 (2006) 9137-9143.

Shen, A. Y., M. H. Hwang, S. Roffler, and C. F. Chen. 1995. Cytotoxicity and antimicrobial activity of some naphthol derivatives. Archiv Pharmazie 328:197-201.

Siddik, Z. H., Cisplatin: mode of cytotoxic action and molecular basis of resistance Oncogene 22 (2003) 7265-7279.

Simon, J. A., and A. Bedalov. 2004. Yeast as a model system for anticancer drug discovery. Nat. Rev. Cancer 4:481-492.

Wilson, G., S. Bentzen, and P. Harari. 2006. Biological basis for combining drugs with radiation. Semin. Radiat. Oncol. 16:2-9.

Yao, S.-L., A. J. Akhtar, K. A. McKenna, G. C. Bedi, D. Sidransky, M. Mabry, R. Ravi, M. I. Collector, R. J. Jones, S. J. Sharkis, E. J. Fuchs, and A. Bedi. 1996. Selective radiosensitization of p53-deficient cells by caffeine-mediated activation of p34cdc2 kinase. Nature Med. 2:1140-1143.

What is claimed is:

1. A pharmaceutical composition comprising a chemotherapeutic agent and an antitumor modulator comprising a bis(3-amino-1-alkanolato-)-di-μ-[halo/hydroxyl/thiocyanato/nitro]-di-Copper complex and derivatives thereof, wherein the chemotherapeutic agent is provided at an optimal or sub-optimal dose.

2. The composition of claim 1, wherein the modulator is selected from Copper, di-μ-chlorobis[1-[(1-piperidinyl-κN)methyl]-2-naphthalenolato-κO]di-(9CI), Copper, di-μ-chlorobis[1-[(4-morpholinyl-κN4)methyl]-2-naphthalenolato-κO]di-(9CI), Copper, di-μ-nitrate bis[1-[(4-morpholinyl-κN4)methyl]-2-naphthalenolato-κO]di-(9CI), Copper, bis(3-amino-1-propanolato-N,O)di-μ-chlorodi-(9CI), Copper, bis(3-amino-1-propanolato-N,O)di-μ-bromodi-(9CI), Copper, bis(3-amino-1-propanolato-N,O)di-μ-hydroxydi-(9CI), Copper, bis[μ-(3-amino-1-propanolato-N,O:O)]dichlorodi-, stereoisomer (9CI), Copper, bis[μ-(3-amino-1-propanolato-N,O:O)]dibromodi-, stereoisomer (9CI), Copper, bis[μ-(3-amino-2-methyl-1-propanolato-N,O:O)]dichlorodi-, stereoisomer (9CI), Copper, bis[μ-(3-amino-2-methyl-1-propanolato-N,O:O)]dichlorodi-, stereoisomer (9CI), Copper, dichlorobis[μ-[3-(methylamino)-1-propanolato-N,O:O]]di-(9CI), Copper, bis(3-amino-1-propanolato-N,O)bis[μ-(thiocyanato-N:S)]di-(9CI), and Copper, bis[μ-(2-aminoethanolato-N,O:O)]dichlorodi-, stereoisomer (9CI).

3. The composition of claim 1, wherein the modulator is in the form of a pharmaceutically acceptable salt thereof.

4. The composition of claim 1, wherein the modulator is in the form of a prodrug thereof.

5. The composition of claim 1, wherein the composition is micronized and is suitable for administration to the warm blooded animal by injection.

6. The composition of claim 1, wherein the composition is administered in an amount of from 10 mg/kg body weight to 10,000 mg/kg body weight.

7. The composition of claim 1, wherein the composition is administered orally, enterically, intravenously, peritoneally, parenterally, subcutaneously, or by injection.

8. The composition of claim 1, wherein the composition is administered in a pharmaceutically acceptable carrier.

9. The composition of claim 1, further comprising a safe and effective amount of a second chemotherapeutic agent.

10. The composition of claim 1, wherein the chemotherapeutic agent is an alkylating agent selected from nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. DNA strand breaking agents include bleomycin, for example. DNA topoisomerase II inhibitors include the following intercalators, such as amsacrine, dactinomycin, daunorubicin, doxorubicin (adriamycin), idarubicin, and mitoxantrone; nonintercalators, such as etoposide and teniposide.

11. The composition of claim 1, wherein the cancer is a carcinoma, leukemia, melanoma, colon cancer, breast cancer, lung cancer, brain cancer, pancreatic cancer, ovarian cancer, head and neck cancer, liver cancer, and prostate cancer.

12. The composition of claim 1, wherein the cancer is selected from the group consisting of astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

13. The composition of claim 1, wherein the bis(3-amino-1-alkanolato-)-di-μ-[halo/hydroxyl/thiocyanato/nitro]-di-Copper complex comprises at least one of:

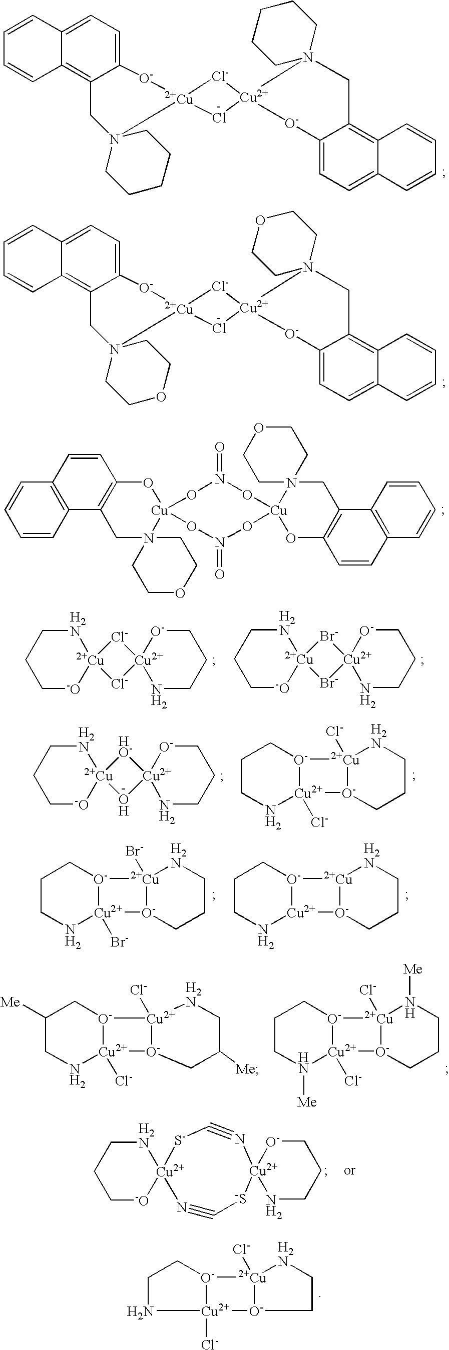

14. A method for treating cancer susceptible to treatment in a warm-blooded animal comprising administering to the warm-blooded animal a therapeutically effective amount of a DNA damaging agent and a therapeutically effective amount of a potentiator of the DNA damaging agent comprising a a bis(3-amino-1-alkanolato-)-di-μ-[halo/hydroxyl/thiocyanato/nitro]-di-Copper complex.

15. The composition of claim 14, wherein the bis(3-amino-1-alkanolato-)-di-μ-[halo/hydroxyl/thiocyanato/nitro]-di-Copper complex comprises at least one of:

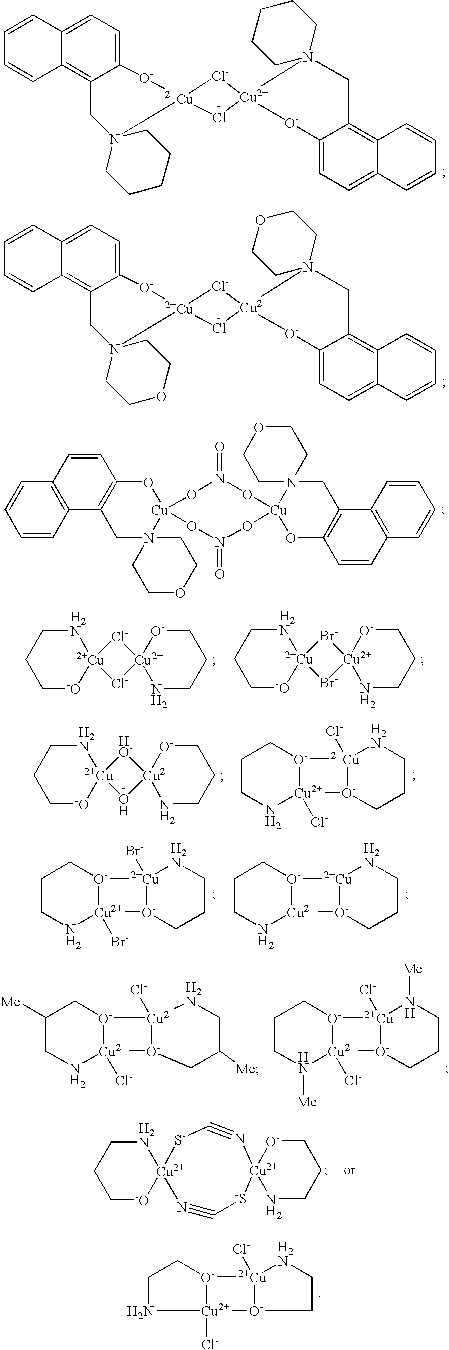

16. The method of claim 14, wherein the modulator is a potentiator in the form of a pharmaceutically acceptable salt thereof.

17. The method of claim 14, wherein the modulator is in the form of a prodrug thereof.

18. The method of claim 14, wherein the composition is micronized and is suitable for administering to the warm blooded animal by injection.

19. The method of claim 14, wherein the composition is administered in an amount of from 10 mg/kg body weight to 10,000 mg/kg body weight.

20. The method of claim 14, wherein the composition is administered orally, enterically, intravenously, peritoneally, parenterally, subcutaneously, or by injection.

21. The method of claim 14, wherein the composition is administered in a pharmaceutically acceptable carrier.

22. The method of claim 14, wherein the cancer is a carcinoma, leukemia, melanoma, colon cancer, breast cancer, lung cancer, brain cancer, pancreatic cancer, ovarian cancer, head and neck cancer, liver cancer, and prostate cancer.

23. The composition of claim 14, wherein the chemotherapeutic agent is an alkylating agent selected from nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. DNA strand breaking agents include bleomycin, for example. DNA topoisomerase II inhibitors include the following intercalators, such as amsacrine, dactinomycin, daunorubicin, doxorubicin (adriamycin), idarubicin, and mitoxantrone; nonintercalators, such as etoposide and teniposide.

24. The method of claim 14, wherein the cancer is selected from the group consisting of astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

25. A method for treating a warm-blooded animal susceptible to DNA damage comprising administering to the warm-blooded animal a therapeutically effective amount of NSC109268.

26. The method of claim 25, wherein the NSC109268 is in the form of a pharmaceutically acceptable or a prodrug thereof.

27. The method of claim 25, wherein the NSC109268 is suitable for administering to the warm blooded animal by injection.

28. The method of claim 25, wherein the NSC109268 is administered in an amount of from 10 mg/kg body weight to 10,000 mg/kg body weight.

29. The method of claim 25, wherein the NSC109268 is administered orally, enterically, intravenously, peritoneally, parenterally, subcutaneously, or by injection.

30. The method of claim 25, wherein the NSC109268 is administered in a pharmaceutically acceptable carrier.

31. The method of claim 25, wherein the DNA damage is caused by a chemical, ultraviolet light, heat, radioactivity, electromagnetic radiation, electricity and combinations thereof.

32. The method of claim 25, wherein the cancer is selected from the group consisting of astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

33. The method of claim 25, wherein the bis(3-amino-1-alkanolato-)-di-μ-[halo/hydroxyl/thiocyanato/nitro]-di-Copper complex comprises an effective dose of at least of:

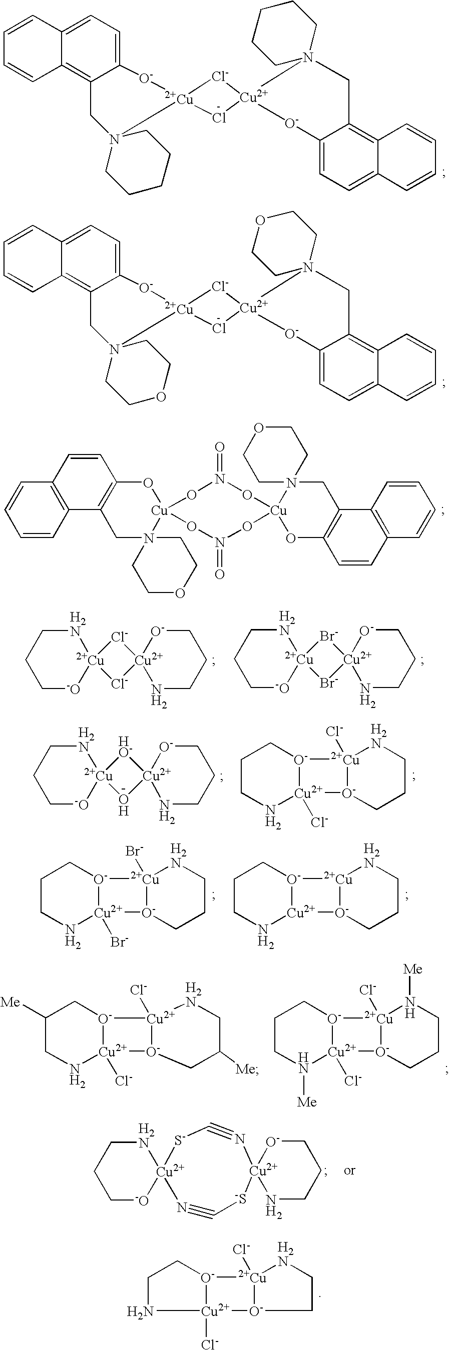

34. A chemotherapeutic comprising a therapeutically effective amount of NSC109268, NSC109272 or both sufficient to treat a cancer cell.

35. The chemotherapeutic of claim 34, wherein the chemotherapeutic a tumor target selected from an astrocytoma, oligodendroglioma, meningioma, neurofibroma, glioblastoma, ependymoma, Schwannoma, neurofibrosarcoma, medulloblastoma, germ cell tumor, chordoma, pineal tumor, choroid plexus papilloma, pituitary tumor, and vascular tumor.

36. The chemotherapeutic of claim 34, further comprising a chemotherapeutic effective amount of a second chemotherapeutic agent.

37. The chemotherapeutic of claim 34, wherein the NSC109268, NSC109272 is adapted for treating a cisplatin-resistant tumor.

38. A method for treating a warm-blooded animal susceptible to DNA damage comprising administering to the warm-blooded animal a therapeutically effective amount of NSC109272.

39. The method of claim 38, wherein the NSC109272 is in the form of a pharmaceutically acceptable or a prodrug thereof.

40. The method of claim 38, wherein the NSC109272 is suitable for administering to the warm blooded animal by injection.

41. The method of claim 38, wherein the NSC109272 is administered in an amount of from 10 mg/kg body weight to 10,000 mg/kg body weight.

42. The method of claim 38, wherein the NSC109272 is administered orally, enterically, intravenously, peritoneally, parenterally, subcutaneously, or by injection.

43. The method of claim 38, wherein the NSC109272 is administered in a pharmaceutically acceptable carrier.
